(19) United States
(12) Patent Application Publication
(10) Pub. No.: US 2023/0372225 A1
(43) Pub. Date: Nov. 23, 2023

(54) TWO-STAGE MAKEUP METHOD

(71) Applicant: L'OREAL, Paris (FR)

(72) Inventors: Grégory PLOS, Chevilly Larue (FR); Philippe ILEKTI, Chevilly Larue (FR); Marion FRAICHE, Chevilly Larue (FR)

(21) Appl. No.: 18/247,085

(22) PCT Filed: Sep. 27, 2021
(86) PCT No.: PCT/EP2021/076457

§ 371 Date: Mar. 29, 2023

(30) Foreign Application Priority Data
Sep. 29, 2020 (FR) FR2009932

(51) Int. Cl. A61K 8/81; A61Q 1/10; A61K 8/55; A61K 8/36

(52) U.S. Cl. CPC A61K 8/817; A61Q 1/10; A61K 8/55; A61K 8/361; A61K 2800/884; A61K 2800/95; A61K 2800/43

(57) ABSTRACT

The present patent application relates to a makeup kit comprising:
a first composition (A) comprising at least 40.0% of water, at least 1.0% by weight, with respect to the total weight of the composition, of cysteine and/or one of its salts, said composition having a pH of greater than 8.0, and
a second composition (B) comprising at least:
i) an aqueous phase; and
ii) a fatty phase comprising at least one wax; and
iii) at least one anionic surfactant comprising at least one cationic counterion, preferably in the form neutralized by said cationic counterion;
iv) optionally at least one film-forming polymer in the form of solid particles in suspension in the aqueous phase of the composition (B).

The invention also relates to a method for making up and in particular for curving said keratin fibers in two stages consisting in successively applying said composition (A) and said composition (B).

The present patent application relates to the field of making up keratin fibers, in particular the eyelashes. It relates more particularly to a makeup kit comprising at least:
a first composition (A) intended to be applied to said keratin fibers comprising, preferably in a physiologically acceptable medium, at least 40.0% of water, with respect to the total weight of the composition, at least 1.0% by weight, with respect to the total weight of the composition, of cysteine and/or of one of its salts, said composition having a pH of greater than 8.0, and
a second composition (B) intended to be applied to the keratin fibers which have been treated by the composition (A), comprising, preferably in a physiologically acceptable medium, at least:
i) an aqueous phase, and
ii) a fatty phase comprising at least one wax, and
iii) at least one anionic surfactant comprising at least one cationic counterion, preferably in the form neutralized by said cationic counterion (salt);
iv) optionally at least one film-forming polymer in the form of solid particles in suspension in the aqueous phase of the composition (B).

The invention also relates to a method for making up and in particular for curving said keratin fibers in two stages consisting in successively applying said composition (A) and said composition (B).

In general, compositions dedicated to making up keratin fibers, such as the eyelashes or the eyebrows, are targeted at densifying the thickness and the visual perception of the eyelashes or eyebrows and ultimately the look. However, another property very particularly desired by consumers is that of obtaining in a significant way a curving of said keratin fibers which is persistent to successive makeup removal operations. Water-resistant anhydrous mascaras do not make it possible to maintain the curvature of the eyelashes after several makeup removal operations.

In the salon, professionals use a makeup method inspired by techniques for the permanent waving of the hair which consists, in a first step, in applying, to the eyelashes, a reducing composition comprising a reducing agent (for example, ammonium thioglycolate and its derivatives, thiol compounds) which breaks the disulfide bridges of the keratin of the eyelashes and then, in a second step, in applying an oxidizing composition comprising at least one oxidizing agent, such as hydrogen peroxide, in order to re-establish the disulfide bridges of the keratin of the eyelashes, shaping the eyelashes and obtaining a durable curving. This method exhibits the disadvantages of being restricting, uncomfortable and not giving the consumer the possibility of easily carrying out this procedure herself at home.

The patent applications KR101823336 and KR101823401 have provided a method for the semipermanent curving of the eyelashes comprising the application, to the eyelashes, of a mascara comprising at least one reducing agent, such as cysteine, cysteamine, thiol compounds, sodium bisulfite, sodium metabisulfite, sodium hydrosulfite or superoxide dismutase, and at least one antioxidant, such as a compound based on phosphoric acid or a compound based on zinc, on gold, on tin or on silver, followed by washing and drying the eyelashes thus treated. This technique for the semipermanent curving of the eyelashes is not entirely satisfactory with regard to the significant curvature effect obtained and with regard to the persistence of the effect after a makeup removal operation.

The need thus remains to find a novel method for making up keratin fibers, in particular the eyelashes, which is easy by its body movements to carry out at home for the consumer and produces an effective curving of said fibers which is persistent, without the constraints of the known methods of the prior art as set out above, such as the use of an oxidizing agent and/or of a shaping support.

Unexpectedly, the inventors have found, during their research studies, that it is possible to achieve this objective with a two-stage makeup method, which consists in successively applying, to said keratin fibers:
a) a first composition (A) comprising, preferably in a physiologically acceptable medium, at least 40.0% by weight of water, with respect to the total weight of the composition, and at least 1.0% by weight, with respect to the total weight of the composition, of cysteine and/or of one of its salts, said composition having a pH of greater than 8.0, and
b) a second composition (B) comprising, preferably in a physiologically acceptable medium:
i) an aqueous phase, and
ii) a fatty phase comprising at least one wax, and
iii) at least one anionic surfactant comprising at least one cationic counterion, preferably in the form neutralized by said cationic counterion (salt),
iv) optionally at least one film-forming polymer in the form of solid particles in suspension in the aqueous phase of the composition (B).

This discovery forms the basis of the invention.

### SUBJECT MATTERS OF THE INVENTION

Thus, a first subject matter of the present invention is a kit for making up keratin fibers, in particular eyelashes, comprising at least:
a) a first composition (A) comprising, preferably in a physiologically acceptable medium, at least 40.0% by weight of water, with respect to the total weight of the composition, and at least 1.0% by weight, with respect to the total weight of the composition, of cysteine and/or of one of its salts, said composition having a pH of greater than 8.0, and
b) a second composition (B) comprising, preferably in a physiologically acceptable medium, at least:
i) an aqueous phase, and
ii) a fatty phase comprising at least one wax, and
iii) at least one anionic surfactant comprising at least one cationic counterion, preferably in the form neutralized by said cationic counterion (salt),
iv) optionally at least one film-forming polymer in the form of solid particles in suspension in the aqueous phase of the composition (B).

A second subject matter of the present invention is a cosmetic method for making up keratin fibers, in particular the eyelashes, comprising:
at least one first stage of application of a first composition (A) as defined above to said keratin substances, and successively
at least one second stage of application of at least one second composition (B) as defined above to the keratin fibers treated by the composition (A).

### Definitions

In the context of the present invention, the term “keratin fibers” is understood to mean in particular the eyelashes and the eyebrows.

The term “physiologically acceptable” is understood to mean compatible with the skin and its superficial body growths, which exhibits a pleasant color and a pleasant feel, and which does not cause any unacceptable discomfort (smarting, tightness) liable to dissuade the consumer from using this composition.

### Reducing Composition (A)

According to the present invention, the reducing composition (A) comprises at least 40.0% by weight of water, with respect to the total weight of the composition, and at least 1.0% by weight, with respect to the total weight of the composition, of cysteine and/or of one of its salts, said composition having a pH of greater than 8.0.

Mention may more particularly be made, among the cosmetically acceptable salts of cysteine, of the hydrochlorides, hydrobromides, citrates, acetates and sulfates.

Preferably, the cysteine employed can be chosen from L-cysteine, D-cysteine or L,D-cysteine. Use will more particularly be made of L-cysteine or one of its salts.

The cysteine and/or one of the salts is preferably present in the composition (A) at a concentration of at least 1% by weight and more preferentially from 1.5% to 8% by weight, with respect to the total weight of the composition (A).

The pH of the reducing composition according to the invention is greater than 8.0 and preferably varies from 8.5 to 10.0.

The pH can be obtained and/or adjusted conventionally by addition of alkaline agents, such as aqueous ammonia, monoethanolamine, diethanolamine, triethanolamine, isopropanolamine, 2-amino-2-methyl-1-propanol, 1,3-propanediamine, an alkali metal or ammonium carbonate or bicarbonate, for example sodium bicarbonate, an organic carbonate, such as guanidine carbonate, or alternatively an alkali metal hydroxide, such as sodium hydroxide or potassium hydroxide, it being possible, of course, for all these compounds to be taken alone or as a mixture.

Use may also be made, in order to obtain and/or adjust, of acidifying agents, such as hydrochloric acid, acetic acid, lactic acid, boric acid, citric acid and phosphoric acid.

Preferably, use will be made of 2-amino-2-methyl-1-propanol or sodium hydroxide.

Use will also be made, in addition to the basifying agent and/or acidifying agent, of a physiologically acceptable buffer, such as a phosphate buffer or a carbonate buffer.

The amount of water is at least 40.0% by weight, with respect to the total weight of the composition. Preferentially, it varies from 50% to 95% by weight and more preferentially from 55% to 95% by weight, with respect to the total weight of the composition (A).

The water can be demineralized, or also a floral water, such as cornflower water, and/or a mineral water, such as Vittel water, Lucas water or La Roche-Posay water, and/or a thermal water.

The composition (A) can contain conventional cosmetic additives, such as hydrophilic gelling agents, preservatives, fragrances, antioxidants, moisturizing agents, such as polyols, for example 1,3-propanediol, glycerol, pentylene glycol, propylene glycol, ethylene glycol, 1,3-butylene glycol, dipropylene glycol and their mixtures, C₃-C₄ ketones, C₂-C₄ aldehydes or C₁-C₅ monoalcohols, such as ethanol or isopropanol.

Of course, a person skilled in the art will take care to choose the optional additional additives and/or their amounts such that the advantageous properties of the composition (A) according to the invention are not, or not substantially, detrimentally affected by the envisaged addition.

### Hydrophilic Gelling Agent

According to a specific form of the invention, the reducing composition (A) according to the invention additionally comprises one or more hydrophilic gelling agent(s).

The term “hydrophilic gelling agent” is understood to mean, within the meaning of the present invention, a compound capable of gelling the aqueous phase of the compositions according to the invention. More particularly, the function held by these hydrophilic gelling agents is to structure the aqueous phase in the composition. The gelling agent is advantageously soluble in the aqueous phase of the composition. The gelling agent which can be used according to the invention can in particular be characterized by its ability to form, in water, beyond a certain concentration C*, a gel characterized by oscillatory rheology (μ=1 Hz) by a yield point Tc at least equal to 10 Pa. This concentration C* can vary widely according to the nature of the gelling agent under consideration.

The hydrophilic gelling agents can be chosen from carboxyvinyl polymers, such as Carbopols® (Carbomers) and Pemulens® (Acrylates/C₁₀-C₃₀ Alkyl Acrylate Copolymer); polyacrylamides, such as, for example, the crosslinked copolymers sold under the names Sepigel 305® (INCI name: Polyacrylamide/C₁₃-C₁₄ Isoparaffin/Laureth 7) or Simulgel 600® (INCI name: Acrylamide/Sodium Acryloyldimethyltaurate Copolymer/Isohexadecane/Polysorbate 80) by SEPPIC; optionally crosslinked and/or neutralized polymers and copolymers of 2-acrylamido-2-methylpropanesulfonic acid, such as poly(2-acrylamido-2-methylpropanesulfonic acid) sold by Hoechst under the trade name “Hostacerin AMPS®” (INCI name: Ammonium Polyacryloyldimethyl Taurate) or Simulgel 800® sold by SEPPIC (INCI name: Sodium Polyacryolyldimethyl Taurate/Polysorbate 80/Sorbitan Oleate); copolymers of 2-acrylamido-2-methylpropanesulfonic acid and of hydroxyethyl acrylate, such as Simulgel NS® and Sepinov EMT 10®, which are sold by SEPPIC; cellulose compounds, such as hydroxyethylcellulose; polysaccharides and in particular gums, such as xanthan gum or hydroxypropylated guar gums.

Preferably, use will be made of cellulose compounds, in particular hydroxyethylcellulose.

The hydrophilic gelling agent(s) are preferably present in the composition in a total content ranging from 0.1% to 10%, better still from 0.5% to 5%, by weight, with respect to the total weight of said composition (A).

### Nonvolatile Oils

According to a specific form of the invention, the reducing composition (A) according to the invention comprises at least one nonvolatile oil.

The term “oil” is understood to mean a water-immiscible nonaqueous compound which is liquid at ambient temperature (25° C.) and atmospheric pressure (760 mmHg).

The term “nonvolatile oil” is understood to mean an oil which remains on the keratin fiber, at ambient temperature and atmospheric pressure, for at least several hours and which has in particular a vapor pressure of less than 10-3 mmHg (0.13 Pa).

These oils can be hydrocarbon oils, silicone oils or their mixtures.

The term “hydrocarbon oil” is understood to mean an oil containing mainly hydrogen and carbon atoms and optionally oxygen, nitrogen, sulfur or phosphorus atoms.

Within the meaning of the present invention, the term “silicone oil” is understood to mean an oil comprising at least one silicon atom, and in particular at least one Si—O group.

Mention may in particular be made, as nonvolatile hydrocarbon oil, of:
hydrocarbon oils of vegetable origin, such as triesters of fatty acids and of glycerol, the fatty acids of which can have varied chain lengths from C₄ to C₂₄, it being possible for these chains to be linear or branched and saturated or unsaturated; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, red kuri squash oil, pumpkin oil, blackcurrant oil, evening primrose oil, millet oil, barley oil, quinoa oil, rye oil, safflower oil, candlenut oil, passionflower oil or musk rose oil; or alternatively triglycerides of caprylic/capric acids, such as those sold by Stéarineries Dubois or those sold under the names Miglyol 810®, 812® and 818® by Dynamit Nobel;
linear or branched hydrocarbons, of mineral or synthetic origin, such as liquid paraffins and their derivatives, polydecenes, polybutenes, hydrogenated polyisobutene, such as Parleam, or squalane;
synthetic ethers having from 10 to 40 carbon atoms;
synthetic esters, such as the oils of formula R₁COOR₂ in which R₁ represents the residue of a linear or branched fatty acid comprising from 1 to 40 carbon atoms and R₂ represents a hydrocarbon chain, in particular branched, containing from 1 to 40 carbon atoms, provided that R₁+R₂ is >10, such as, for example, purcellin oil (cetostearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, alkyl or polyalkyl octanoates, decanoates or ricinoleates, such as propylene glycol dioctanoate; hydroxylated esters, such as isostearyl lactate or diisostearyl malate; and pentaerythritol esters;
fatty alcohols which are liquid at ambient temperature, comprising a branched and/or unsaturated carbon chain having from 12 to 26 carbon atoms, such as octyldodecanol, isostearyl alcohol, oleyl alcohol, 2-hexyldecanol, 2-butyloctanol or 2-undecylpentadecanol;
higher fatty acids, such as oleic acid, linoleic acid, linolenic acid; and their mixtures.

The nonvolatile silicone oils which can be used in the composition according to the invention can be nonvolatile polydimethylsiloxanes (PDMSs), polydimethylsiloxanes comprising alkyl or alkoxy groups, which groups are pendent and/or at the end of the silicone chain and each have from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyldiphenyl)trisiloxanes and (2-phenylethyl)trimethylsiloxysilicates.

According to a preferential form of the invention, the nonvolatile oil or oils are present in the reducing composition (A) in a total concentration of less than 5.0% by weight, with respect to the total weight of the composition (A). In a preferred embodiment, the composition (A) is free from nonvolatile oil.

### Shaping Composition (B)

The composition (B) according to the invention for shaping the keratin fibers comprises, preferably, in a physiologically acceptable medium:
i) an aqueous phase, and
ii) a fatty phase comprising at least one wax, and
iii) at least one anionic surfactant comprising at least one cationic counterion, preferably in the form neutralized by said cationic counterion (salt),
iv) optionally at least one film-forming polymer in the form of solid particles in suspension in the aqueous phase of the composition (B).

### Aqueous Phase

The composition (B) according to the invention comprises an aqueous phase.

The term “aqueous phase” is understood to mean a phase comprising water and also optionally all the solvents and ingredients which are water-soluble or water-miscible (water-miscibility of greater than 50% by weight at 25° C.), such as lower monoalcohols having from 1 to 5 carbon atoms, such as ethanol or isopropanol, polyols having from 3 to 8 carbon atoms, such as propylene glycol, 1,3-butylene glycol, caprylyl glycol, pentylene glycol, glycerol or dipropylene glycol, C₃-C₄ ketones or C₂-C₄ aldehydes.

The aqueous phase can contain a demineralized water, or also a floral water, such as cornflower water, and/or a mineral water, such as Vittel water, Lucas water or La Roche-Posay water, and/or a thermal water.

The water is present, preferably, in a concentration ranging from 30% to 70% by weight, with respect to the total weight of the composition (B), preferably ranging from 40% to 60% by weight, with respect to the total weight of the composition, preferably ranging from 45% to 55% by weight, with respect to the total weight of the composition (B).

### Fatty Phase

The shaping composition (B) according to the invention comprises a fatty phase, which is preferably a dispersed fatty phase.

The fatty phase can be present in a content ranging from 5% to 45% by weight, preferably from 10% to 45% by weight, better still from 10% to 40% by weight, with respect to the total weight of the composition (B).

The term “fatty phase” is understood to mean a phase comprising at least one fatty substance, such as oils and waxes, preferably waxes, and also optionally all the organic solvents and ingredients which are soluble or miscible in said phase when it is in the liquid state and, if said fatty phase becomes solid again at ambient temperature, said organic solvents and ingredients give a macroscopically homogeneous mixture.

According to the present invention, the fatty phase of the composition (B) comprises at least one wax.

### Waxes

Preferably, the composition(s) according to the invention comprise(s) at least one wax. This wax makes it possible to obtain thick and charging textures.

The term “wax” is understood to mean a lipophilic compound which is solid at ambient temperature (25° C.), which is or is not deformable, which exhibits a reversible solid/liquid change in state and which has a melting point of greater than or equal to 40° C. which can range up to 120° C. In particular, the waxes suitable for the invention can exhibit a melting point of greater than or equal to 45° C. and in particular of greater than or equal to 55° C.

The term “lipophilic compound” is understood to mean a compound having an acid number and a hydroxyl number of less than 150 mg KOH/g.

Within the meaning of the invention, the melting point corresponds to the temperature of the most endothermic peak observed in thermal analysis (DSC) as described in the standard ISO 11357-3:1999. The melting point of the wax can be measured using a differential scanning calorimeter (DSC), for example the calorimeter sold under the name MDSC 2920® by TA Instruments.

The measurement protocol is as follows:

A sample of 5 mg of wax placed in a crucible is subjected to a first temperature rise ranging from −20° C. to 100° C., at a heating rate of 10° C./minute, is then cooled from 100° C. to −20° C. at a cooling rate of 10° C./minute and is finally subjected to a second temperature rise ranging from −20° C. to 100° C. at a heating rate of 5° C./minute. During the second temperature rise, the variation in the difference in power absorbed by the empty crucible and by the crucible containing the sample of wax is measured as a function of the temperature. The melting point of the compound is the value of the temperature corresponding to the tip of the peak of the curve representing the variation in the difference in power absorbed as a function of the temperature.

The waxes can be hydrocarbon waxes, silicone waxes and/or fluoro waxes, and can be of vegetable, mineral and/or synthetic origin.

The wax(es) are present, preferably, in a content of at least 5% by weight, more preferentially in a content ranging from 5% to 45% by weight, with respect to the total weight of composition, better still ranging from 8% to 40% and even better still from 10% to 40% by weight, with respect to the total weight of composition (B).

Use may in particular be made, as wax, of hydrocarbon waxes, such as beeswax, lanolin wax; rice wax, carnauba wax, candelilla wax, ouricury wax, Japan wax, berry wax, shellac wax and sumac wax; montan wax.

Mention may also be made of the waxes obtained by catalytic hydrogenation of animal or vegetable oils having linear or branched C₈-C₃₂ fatty chains.

Mention may in particular be made, among these waxes, of hydrogenated jojoba oil, hydrogenated palm oil, hydrogenated sunflower oil, hydrogenated castor oil, hydrogenated coconut oil and hydrogenated lanolin oil, bis(1,1,1-trimethylolpropane) tetrastearate sold under the name Hest 2T-4S® by Heterene or bis(1,1,1-trimethylolpropane) tetrabehenate sold under the name Hest 2T-4B® by Heterene.

Use may also be made of the wax obtained by hydrogenation of olive oil esterified with stearyl alcohol, sold under the name Phytowax Olive 18 L 57®, or alternatively the waxes obtained by hydrogenation of castor oil esterified with cetyl alcohol, which are sold under the names Phytowax Ricin 16L64® and 22L73® by Sophim. Such waxes are described in the application FR-A-2 792 190.

Use may also be made of a C₂₀-C₄₀ alkyl (hydroxystearyloxy)stearate (the alkyl group comprising from 20 to 40 carbon atoms), alone or as a mixture, in particular a C₂₀-C₄₀ alkyl 12-(12′-hydroxystearoyloxy)stearate, of formula (I):

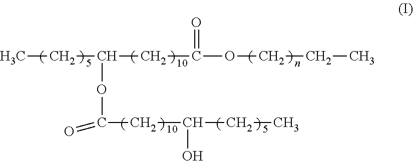

in which n is an integer ranging from 18 to 38, or a mixture of compounds of formula (I). Such a tacky wax is in particular sold under the names Kester Wax K 82 P® and Kester Wax K 80 P® by Koster Keunen.

Mention may be made of microcrystalline waxes, paraffins and ozokerite, polyethylene waxes, the waxes obtained by the Fisher-Tropsch synthesis and waxy copolymers, and also their esters; silicone waxes and fluoro waxes.

Mention may be made of the linear fatty acid monoesters of following formula (I):

[Chem 2]

R₃—O—R₄ (1)
in which R₃ and R₄ are linear and saturated and have, independently of each other, a number of carbon atoms of greater than or equal to 20, with R₃ representing an acyl radical and R₄ representing an alkyl radical.

In particular, the fatty acid monoester according to the invention is chosen from arachidyl arachidate and behenyl behenate and more particularly behenyl behenate.

According to a preferred mode, use will be made of a wax chosen from beeswax, carnauba wax, cetyl alcohol, paraffins, behenyl behenate and their mixtures.

The fatty phase of the composition (B) can also contain, in addition, at least one nonvolatile oil, such as those used in the reducing composition (A).

The nonvolatile oil or oils are present in the composition (B) in amounts preferably of less than 5% by weight, with respect to the total weight of the composition (B). The composition (B) is, according to a preferred embodiment, free from nonvolatile oil.

### Anionic Surfactant

The composition (B) according to the invention comprises at least one anionic surfactant comprising at least one cationic counterion, preferably at least one anionic surfactant in the form neutralized by said cationic counterion (salt).

Within the meaning of the present invention, the term “surfactant” is understood to mean an amphiphilic chemical compound, that is to say a compound exhibiting, in its structure, two parts of different polarity. Generally, one is lipophilic (soluble or dispersible in an oily phase). The other is hydrophilic (soluble or dispersible in water). Surfactants are characterized by the value of their HLB (hydrophilic-lipophilic balance), the HLB being the ratio of the hydrophilic part to the lipophilic part in the molecule. The term “HLB” is well known to a person skilled in the art and is described, for example, in “The HLB System. A Time-Saving Guide to Emulsifier Selection” (published by ICI Americas Inc.; 1984).

According to a preferential form of the invention, the anionic surfactants comprising at least one cationic counterion in accordance with the invention have an HLB of greater than or equal to 8.0, indeed even preferentially of greater than 10.0. The HLB of the anionic surfactant(s) used according to the invention can be determined by the Griffin method.

The term “anionic surfactant” is understood to mean any negatively charged amphiphilic molecule.

The term “cationic counterion” is understood to mean any positively charged ion or ionic group capable of neutralizing said anionic surfactant and of forming a salt.

The anionic surfactant(s) in accordance with the invention is (are) preferably chosen from:
i. alkyl phosphates;
ii. alkyl sulfates, and in particular alkyl ether sulfates, alkylamido ether sulfates, alkylaryl polyether sulfates, monoglyceride sulfates;
iii. alkylsulfonates, alkylamidesulfonates, alkylarylsulfonates, α-olefinsulfonates, paraffinsulfonates;
iv. alkyl sulfosuccinates, alkyl ether sulfosuccinates, alkylamide sulfosuccinates; alkyl sulfosuccinamates;
v. alkyl sulfoacetates;
vi. acyl sarcosinates, acyl glutamates, acyl isethionates, N-acyl taurates, acyl lactylates;
vii. alkylpolyglycoside carboxylic esters, such as alkylpolyglucoside citrates, alkylpolyglycoside tartrates and alkylpolyglycoside sulfosuccinates, alkylpolyglycoside sulfosuccinamates;
viii. fatty acids, in particular oleic acid, ricinoleic acid, palmitic acid or stearic acid, coconut oil acid or hydrogenated coconut oil acid;
ix. alkyl-D-galactosiduronic acids;
x. polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids, in particular those comprising from 2 to 50 alkylene oxide, in particular ethylene oxide, groups;
xi. their mixtures.

The anionic surfactant(s) in accordance with the invention is (are) preferably present at a total content of greater than or equal to 2% by weight, with respect to the total weight of the composition, preferably ranging from 2% to 15%, better still from 3% to 8%, by weight, with respect to the total weight of the composition (B).

The cationic counterion(s) in accordance with the invention is (are) chosen from a cation of inorganic origin, in particular chosen from alkali metal (sodium, potassium) cations, the ammonium ion (NH₄+) or organic cations.

Said organic cationic counterion(s) is (are) preferably chosen from amines and aminoalcohols.

According to a specific form of the invention, said organic cationic counterion(s) comprise(s) a primary (poly)hydroxyalkylamine.

The term “primary (poly)hydroxyalkylamine” is understood to mean in particular a primary dihydroxyalkylamine, it being understood that the term “primary” is understood to mean a primary amine, i.e. —NH₂, functional group, and the alkyl group being a linear or branched C₁-C₈, preferably branched C₄, hydrocarbon chain, such as 1,3-dihydroxy-2-methylpropyl (also known as aminomethylpropanediol or AMPD).

The total content of cationic counterion(s) is preferably greater than or equal to 0.01%, more preferentially inclusively between 0.1% and 4.0% by weight, better still between 0.5% and 3.0% by weight, with respect to the total weight of the composition.

According to a specific form of the invention, said anionic surfactant(s) is (are) chosen from:
C₁₂-C₂₂, preferably C₁₄-C₁₈, fatty acids, more particularly the salt of stearic acid and of the cationic counterion aminomethylpropanediol;
alkyl phosphates, preferably in the form neutralized by a cationic counterion of an alkali metal and more particularly potassium. Mention may be made, by way of example, of the anionic surfactant in the form neutralized by its potassium cationic counterion with the INCI name: Potassium Cetyl Phosphate, such as the commercial product sold under the name Amphisol K® by DSM Nutritional Products Europe Ltd;
their mixtures.

### Film-Forming Polymer in Aqueous Suspension

According to a specific form of the present invention, the shaping composition (B) comprises at least one film-forming polymer in the form of solid particles in suspension in the aqueous phase of the composition.

In the present patent application, the term “film-forming polymer” is understood to mean a polymer capable of forming, by itself alone or in the presence of an auxiliary film-forming agent, a continuous deposit on a support, at a temperature of 20° C.

Said solid particles of film-forming polymer can either be used as is and are in suspension in the aqueous phase of the composition or can be used in the form of particles in aqueous dispersion (latex or pseudolatex).

Such a film-forming polymer present in the form of particles in aqueous dispersion is generally named (pseudo)latex, that is to say latex or pseudolatex. The techniques for the preparation of these dispersions are well known to a person skilled in the art.

The composition (B) according to the invention can comprise one or more types of particles, it being possible for these particles to vary by their size, by their structure and/or by their chemical nature.

The film-forming polymer(s) can be present in a solids content ranging from 0.5% to 10% by weight, with respect to the total weight of the composition, preferably ranging from 1% to 5% by weight, with respect to the total weight of the composition (B).

These solid particles can be of anionic, cationic or neutral nature and can constitute a mixture of solid particles of different natures.

In the present invention, the term “aqueous” is understood to mean a liquid medium based on water and/or on hydrophilic solvents. This aqueous liquid medium can be composed essentially of water. It can also comprise a mixture of water and of water-miscible organic solvent(s) (miscibility in water of greater than 50% by weight at 25° C.), such as lower monoalcohols having from 2 to 5 carbon atoms, such as ethanol or isopropanol, glycols having from 3 to 8 carbon atoms, such as propylene glycol, 1,3-butylene glycol or dipropylene glycol, C₃-C₄ ketones or C₂-C₄ aldehydes.

Mention may be made, among the film-forming polymers which can be used in the composition of the present invention, of synthetic polymers of radical or of polycondensate type, polymers of natural origin and their mixtures. In general, these polymers can be random polymers, block copolymers of type A-B, multiblocks A-B-A or also ABCD, and the like, indeed even grafted polymers.

### Radical Film-Forming Polymer

The term “radical polymer” is understood to mean a polymer obtained by polymerization of unsaturated monomers, in particular ethylenically unsaturated monomers, each monomer being capable of homopolymerizing (unlike polycondensates).

The film-forming polymers of radical type can in particular be acrylic and/or vinyl homopolymers or copolymers.

The vinyl film-forming polymers can result from the polymerization of ethylenically unsaturated monomers having at least one acid group and/or of the esters of these acid monomers and/or of the amides of these acid monomers.

Use may be made, as ethylenically unsaturated monomers having at least one acid group or monomer carrying an acid group, of α,β-ethylenic unsaturated carboxylic acids, such as acrylic acid, methacrylic acid, crotonic acid, maleic acid or itaconic acid. Use is made in particular of (meth)acrylic acid and crotonic acid, and more particularly of (meth)acrylic acid.

The esters of acid monomers are advantageously chosen from esters of (meth)acrylic acid (also known as (meth)acrylates), in particular alkyl, especially C₁-C₂₀ alkyl, more particularly C₁-C₈ alkyl, (meth)acrylates, aryl, in particular C₆-C₁₀ aryl, (meth)acrylates or hydroxyalkyl, in particular C₂-C₆ hydroxyalkyl, (meth)acrylates.

Mention may be made, among the alkyl (meth)acrylates, of methyl methacrylate, ethyl methacrylate, butyl methacrylate, isobutyl methacrylate, 2-ethylhexyl methacrylate or lauryl methacrylate.

Mention may be made, among the hydroxyalkyl (meth)acrylates, of hydroxyethyl acrylate, 2-hydroxypropyl acrylate, hydroxyethyl methacrylate or 2-hydroxypropyl methacrylate.

Mention may be made, among the aryl (meth)acrylates, of benzyl acrylate and phenyl acrylate.

The esters of (meth)acrylic acid are in particular alkyl (meth)acrylates.

According to the present invention, the alkyl group of the esters can be either fluorinated or perfluorinated, that is to say that some or all of the hydrogen atoms of the alkyl group are replaced by fluorine atoms.

Mention may be made, as amides of the acid monomers, for example, of (meth)acrylamides, and in particular N-alkyl(meth)acrylamides, especially N—(C₂-C₁₂ alkyl)(meth)acrylamides. Mention may be made, among the N-alkyl(meth)acrylamides, of N-ethylacrylamide, N-(t-butyl)acrylamide and N-(t-octyl)acrylamide.

The vinyl film-forming polymers can also result from the homopolymerization or copolymerization of monomers chosen from vinyl esters and styrene monomers. In particular, these monomers can be polymerized with acid monomers and/or their esters and/or their amides, such as those mentioned above.

Mention may be made, as examples of vinyl esters, of vinyl acetate, vinyl neodecanoate, vinyl pivalate, vinyl benzoate and vinyl t-butylbenzoate.

Mention may be made, as styrene monomers, of styrene and α-methylstyrene.

The list of the monomers given is not limiting and it is possible to use any monomer known to a person skilled in the art falling in the categories of acrylic and vinyl monomers (including monomers modified by a silicone chain).

Mention may also be made of polymers resulting from the radical polymerization of one or more radical monomers inside and/or partially at the surface of pre-existing particles of at least one polymer chosen from the group consisting of polyurethanes, polyureas, polyesters, polyesteramides and/or alkyds. These polymers are generally referred to as “hybrid polymers”.

### Polycondensate

Mention may be made, as film-forming polymer of polycondensate type, of polyurethanes which are anionic, cationic, nonionic or amphoteric, polyurethanes-acrylics, polyurethanes-polyvinylpyrrolidones, polyesters-polyurethanes, polyethers-polyurethanes, polyureas, polyureas-polyurethanes, silicone polyurethanes and their mixtures.

The film-forming polyurethane can, for example, be an aliphatic, cycloaliphatic or aromatic polyurethane, polyurea/urethane or polyurea copolymer comprising, alone or as a mixture, at least one block chosen from:
a block of aliphatic and/or cycloaliphatic and/or aromatic polyester origin, and/or
a branched or unbranched silicone block, for example polydimethylsiloxane or polymethylphenylsiloxane, and/or
a block comprising fluoro groups.

The film-forming polyurethanes as defined in the invention can also be obtained from branched or unbranched polyesters or from alkyds comprising mobile hydrogens, which are modified by reaction with a diisocyanate and a difunctional organic compound (for example dihydro, diamino or hydroxyamino), additionally comprising either a carboxylic acid or carboxylate group, or a sulfonic acid or sulfonate group, or alternatively a neutralizable tertiary amine group or a quaternary ammonium group.

Mention may also be made, among the film-forming polycondensates, of polyesters, polyesteramides, fatty-chain polyesters, polyamides and epoxyester resins.

The polyesters can be obtained, in a known way, by polycondensation of dicarboxylic acids with polyols, in particular diols.

The dicarboxylic acid can be aliphatic, alicyclic or aromatic. Mention may be made, as examples of such acids, of: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers can be used alone or as a combination of at least two dicarboxylic acid monomers. The choice is made in particular, among these monomers, of phthalic acid, isophthalic acid and terephthalic acid.

The diol can be chosen from aliphatic, alicyclic or aromatic diols. Use is made in particular of a diol used chosen from: ethylene glycol, diethylene glycol, triethylene glycol, 1,3-propanediol, cyclohexanedimethanol or 1,4-butanediol. Use may be made, of other polyols, of glycerol, pentaerythritol, sorbitol or trimethylolpropane.

The polyesteramides can be obtained analogously to the polyesters, by polycondensation of diacids with diamines or aminoalcohols. Use may be made, as diamine, of ethylenediamine, hexamethylenediamine or meta- or para-phenylenediamine. Use may be made, as aminoalcohol, of monoethanolamine.

Use will be made, as specific form of polycondensate film-forming polymer, of a polyester-polyurethane in aqueous dispersion with the INCI name: Polyurethane-35, such as the commercial product Baycusan C 1010/1®, and/or that with the INCI name: Polyurethane-48, such as the commercial product Baycusan C 1008/1®, which are sold by Covestro.

### Polymer of Natural Origin

Use may be made, in the present invention, of optionally modified polymers of natural origin, such as shellac resin, sandarac gum, dammars, elemis, copals, water-insoluble cellulose-based polymers, such as nitrocellulose, modified cellulose esters, including in particular carboxyalkyl cellulose esters, such as those described in the patent application US2003/185774, and their mixtures.

According to a specific embodiment of the invention, said at least one film-forming polymer in the dispersed state is chosen from acrylic polymer dispersions, polyurethane dispersions, sulfopolyester dispersions, vinyl dispersions, polyvinyl acetate dispersions, vinylpyrrolidone, dimethylaminopropylmethacrylamide and lauryldimethylmethacrylamidopropylammonium chloride terpolymer dispersions, dispersions of polyurethane/polyacrylic hybrid polymers, dispersions of particles of core-shell type and their mixtures.

Various types of aqueous dispersions, in particular commercial aqueous dispersions, which are suitable for the preparation of the composition in accordance with the present invention, are described in detail below.

1/ Thus, according to a preferred embodiment of the invention, the aqueous dispersion of polymer particles is an aqueous dispersion of acrylic polymer.

The acrylic polymer can be a styrene/acrylate copolymer and in particular a polymer chosen from the copolymers resulting from the polymerization of at least one styrene monomer and at least one C₁-C₁₈ alkyl (meth)acrylate monomer. Mention may be made, as styrene monomer which can be used in the invention, for example, of styrene or α-methylstyrene, and in particular styrene.

The C₁-C₁₈ alkyl (meth)acrylate monomer is in particular a C₁-C₁₂ alkyl (meth)acrylate and more particularly a C₁-C₁₀ alkyl (meth)acrylate. The C₁-C₁₈ alkyl (meth)acrylate monomer can be chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate.

Use may be made according to the invention, as acrylic polymer in aqueous dispersion, of:
the aqueous dispersions of acrylic polymer which are sold under the names Acronal DS-6250® by BASF, Neocryl A-45®, Neocryl XK-90®, Neocryl A-1070®, Neocryl A-1090®, Neocryl BT-62®, Neocryl A-1079® and Neocryl A-523® by DSM, Joncryl 95® or Joncryl 8211® by BASF, Daitosol 5000 AD® (INCI name: Acrylates Copolymer) or Daitosol 5000 SJ® (INCI name: Acrylates/Ethylhexyl Acrylate Copolymer) by Daito Kasei Kogyo; Syntran 5760® (INCI name: Styrene/Acrylates/Ammonium Methacrylate Copolymer) by Interpolymer.

2/ According to an alternative embodiment of the invention, the aqueous dispersion of polymer particles is an aqueous dispersion of particles of polyester-polyurethane and/or polyether-polyurethane which is in particular anionic.

The anionic nature of the polyester-polyurethanes and of the polyether-polyurethanes used according to the invention is due to the presence in their constituent units of groups having a carboxylic acid or sulfonic acid functional group.

The polyester-polyurethane or polyether-polyurethane particles used according to the invention are generally sold in the form of aqueous dispersions.

The content of particles of said dispersions currently available on the market ranges from approximately 20% to approximately 50% by weight, with respect to the total weight of the dispersion.

Mention may in particular be made, among the anionic polyester-polyurethane dispersions which can be used in the aqueous varnishes according to the invention, of that sold under the name Avalure UR 405® by Noveon.

Mention may in particular be made, among the anionic polyether-polyurethane particle dispersions which can be used according to the invention, of those sold under the name Avalure UR 450® by Noveon and under the name Neorez R 970® by DSM, Baycusan C 1010® (INCI name: Polyurethane-35), such as the product sold under the name Baycusan C 1010/1®, and Baycusan C 1008/1® (INCI name: Polyurethane-48 (and) Methylpropanediol (and) Caprylyl Glycol (and) Phenylpropanol), which are sold by Covestro.

According to a specific embodiment of the invention, use may be made of a mixture of commercial dispersions consisting of anionic polyester-polyurethane particles as are defined above and of anionic polyether-polyurethane particles which are also defined above.

For example, use may be made of a mixture consisting of the dispersion sold under the name Sancure 861® or a mixture of the dispersion sold under the name Avalure UR 405® and of the dispersion sold under the name Avalure UR 450®, these dispersions being sold by Noveon.

Mention may also be made, as other examples of film-forming polymers in accordance with the invention, of:
the polycondensates of polyamide as described in the documents U.S. Pat. Nos. 5,874,069, 5,919,441, 6,051,216 and 5,981,680, such as, for example, those sold under the references Dow Corning 2-8179® and Dow Corning 2-8178 Gellant® by Dow Corning; the sequential copolymers of “diblock”, “triblock” or “radial” type of the polystyrene/polyisoprene, polystyrene/polybutadiene type, such as those sold under I M 5960®).

Use may more preferentially be made, as solid particles of film-forming polymer according to the invention, of:
aqueous dispersions of acrylic polymer which are sold under the names Acronal DS-6250® by BASF, Neocryl A-45®, Neocryl XK-90®, Neocryl A-1070®, Neocryl A-1090®, Neocryl BT-62®, Neocryl A-1079® and Neocryl A-523® by DSM, Joncryl 95® or Joncryl 8211® by BASF, Daitosol 5000 AD® (INCI name: Acrylates Copolymer) or Daitosol 5000 SJ® (INCI name: Acrylates/Ethylhexyl Acrylate Copolymer) by Daito Kasey Kogyo, Acudyne 5600P® and Acudyne 5800P® by Dow Chemical (INCI name: Acrylates Copolymer);
the aqueous dispersions of polyurethane which are sold under the names Neorez R-981® and Neorez R-974® by DSM, Avalure UR-405®, Avalure UR-410®, Avalure UR-425®, Avalure UR-450®, Sancure 875®, Avalure UR 445® or Avalure UR 450® by Noveon, Impranil 85® by Bayer, Baycusan C 1010® (INCI name: Polyurethane-35), such as the product sold under the name Baycusan C 1010/1®, and Baycusan C 1008/1® (INCI name: Polyurethane-48 (and) Methylpropanediol (and) Caprylyl Glycol (and) Phenylpropanol), which are sold by Covestro.
sulfopolyesters, such as those sold under the brand name Eastman AQ® by Eastman Chemical Products,
vinyl dispersions, such as Mexomere PAM®, aqueous dispersions of polyvinyl acetate, such as Vinybran® from Nisshin Chemical or those sold by Union Carbide, aqueous dispersions of vinylpyrrolidone, dimethylaminopropylmethacrylamide and lauryldimethylmethacrylamidopropylammonium chloride terpolymer, such as Styleze W® from ISP,
aqueous dispersions of polyurethane/polyacrylic hybrid polymers, such as those sold under the references Hybridur® by Air Products or Duromer® by National Starch,
their mixtures.

According to a preferred embodiment, a composition (B) in accordance with the invention is an aqueous dispersion of acrylic film-forming polymer.

According to a specific form, a composition (B) in accordance with the invention comprises an aqueous dispersion of solid particles of film-forming polymer of styrene, of ammonium methacrylate and of a monomer chosen from acrylic acid, methacrylic acid or one of their simple esters with the INCI name: Styrene/Acrylates/Ammonium Methacrylate Copolymer. Mention may be made, by way of examples, of the commercial products Syntran 5760 CG®, Syntran 5620 CG® and Syntran PC 5610® sold by Interpolymer.

According to a specific form, a composition (B) in accordance with the invention comprises an aqueous dispersion of solid particles of film-forming polymer of styrene, of ammonium methacrylate and of a monomer chosen from acrylic acid, methacrylic acid or one of their simple esters with the INCI name: Styrene/Acrylates/Ammonium Methacrylate Copolymer, and an aqueous dispersion of solid particles of polyester-polyurethane with the INCI name: Polyurethane-35, such as the commercial product sold under the name Baycusan C 1010/1® by Covestro.

### Colorant

According to a specific form of the invention, the shaping composition (B) according to the invention additionally comprises at least one colorant, in particular chosen from pulverulent colorants, water-soluble dyes and their mixtures.

The pulverulent colorants can be chosen from pigments and pearlescent agents.

The pigments can be white or colored, inorganic and/or organic, and coated or uncoated. Mention may be made, among the inorganic pigments, of titanium dioxide, which is optionally surface-treated, zirconium oxide, zinc oxide or cerium oxide, and also iron oxide, chromium oxide, manganese violet, ultramarine blue, chromium hydrate and ferric blue. Mention may be made, among the organic pigments, of carbon black, pigments of D & C type, and lakes based on cochineal carmine and on barium, strontium, calcium or aluminum.

The pearlescent agents can be chosen from white pearlescent pigments, such as mica coated with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and also pearlescent pigments based on bismuth oxychloride.

Preferably, the composition according to the invention comprises a pulverulent colorant, preferably of pigment type, in particular metal oxides, especially titanium dioxides, iron oxides and their mixtures; and more particularly iron oxides.

Mention may in particular be made, as water-soluble dyes suitable for the invention, of synthetic or natural water-soluble dyes, such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophyllin, methylene blue, anthocyanins (enocyanin, black carrot, hibiscus or elder), caramel or riboflavin.

The water-soluble dyes are, for example, beetroot juice and caramel.

Preferably, said colorant is present in the composition (B) in a content ranging from 2.0% to 25.0% by weight, preferably from 3.0% to 20.0% by weight, more particularly from 4.0% to 15.0% by weight, with respect to the total weight of the composition (B).

### Additives

Mention may in particular be made, as cosmetic additives which can be used in the compositions (B) according to the invention, of antioxidants; preservatives; fragrances; organic or inorganic fillers; sequestering agents; chelating agents; sunscreens; vitamins, such as vitamin C or ascorbic acid and its derivatives, such as 2-O-alpha-D-glucopyranosyl-L-ascorbic acid (INCI name: Ascorbyl Glucoside), vitamin B3 or niacinamide; vitamin B8, also called H: D-Biotin, vitamin E and its derivatives, such as tocopherol acetate; and their mixtures.

These additives can be present in the composition (B) in a content ranging from 0.01% to 15.0% of the total weight of the composition (B).

Of course, a person skilled in the art will take care to choose the optional additional additives and/or their amounts such that the advantageous properties of the composition according to the invention are not, or not substantially, detrimentally affected by the envisaged addition.

According to a specific form of the invention, the composition (B) is in the form of a product for the eyelashes, such as a mascara.

### Method for Making Up Keratin Fibers

A subject matter of the present patent application is a cosmetic method for making up keratin fibers, in particular the eyelashes, comprising
at least one first stage of application of a first composition (A) as defined above to said keratin fibers, and successively
at least one second stage of application, to said fibers treated by the composition (A), of at least one second composition (B) as defined above.

Between the application of the first reducing composition (A) and the application of the second shaping composition (B), it is possible to provide a leave-in time preferably varying from 1 to 20 minutes, more preferentially from 2 to 15 minutes, and a stage of wiping off the surplus of composition (A), before applying the composition (B). The wiping off of the surplus of the composition (A) can be carried out using a cotton swab, a disk of makeup-removing cotton or also absorbent paper, or paper towels. It is possible to provide a contact time between the eyelash and the shaping composition (B) for several hours, such as, for example, 4 hours.

Preferentially, in order to increase the curving of the keratin fibers and the persistence of the effect, in particular to makeup removal, the makeup method of the invention is repeated several times, preferably 3 times, successively or at various moments separated in time, by successively applying the compositions (A) and (B).

The preferred embodiment consists in applying, once per day, the method of the invention with the compositions (A) and (B), three days in a row.

### Packagings and Applicators

The compositions (A) and (B) according to the invention can each be packaged in a container delimiting at least one compartment which comprises said composition (A) or (B), said container being closed by a closing element.

The container can be in any suitable form. It can in particular be in the form of a bottle, a tube, a jar or a case.

The closing element may be in the form of a removable stopper, a lid or a cover, especially of the type comprising a body fixed to the container and a cap articulated on the body. It can also be in the form of an element ensuring the selective closure of the container, in particular a pump, a valve or a shutter.

The container can be combined with an applicator, in particular in the form of a brush comprising an arrangement of bristles held by a twisted wire. Such a twisted brush is described in particular in the patent U.S. Pat. No. 4,887,622. It can also be in the form of a comb comprising a plurality of application elements, which are obtained in particular by molding. Such combs are described, for example, in the patent FR 2 796 529. The applicator can be in the form of a fine brush, such as described, for example, in the patent FR 2 722 380. The applicator can be in the form of a block of foam or of elastomer. The applicator can be free (sponge) or integral with a rod carried by the closing element, such as described, for example, in the patent U.S. Pat. No. 5,492,426. The applicator can be integral with the container, such as described, for example, in the patent FR 2 761 959.

The product can be contained directly in the container, or indirectly.

The closing element can be coupled to the container by screwing. Alternatively, the coupling between the closing element and the container is done other than by screwing, in particular via a bayonet mechanism, by click-fastening or by gripping. The term “click-fastening” is understood in particular to mean any system involving the crossing of a bead or cord of material by elastic deformation of a portion, in particular of the closing element, followed by return to the elastically unconstrained position of said portion after the crossing of the bead or cord.

The container can be at least partially made of thermoplastic material. Mention may be made, as examples of thermoplastic materials, of polypropylene or polyethylene.

The container can have rigid or deformable walls, in particular in the form of a tube or a tube bottle.

The container can comprise means intended to bring about or facilitate the dispensing of the composition. By way of example, the container can have deformable walls, so as to bring about the departure of the composition in response to an excess pressurization inside the container, which excess pressurization is brought about by the elastic (or nonelastic) crushing of the walls of the container.

The container can be equipped with a drainer positioned in the vicinity of the opening of the container. Such a drainer makes it possible to wipe the applicator and optionally the rod to which it may be integrally attached. Such a drainer is described, for example, in the patent FR 2 792 618.

Throughout the description, including the claims, the expression “comprising a” should be understood as being synonymous with “comprising at least one”, unless otherwise specified.

The expressions “between . . . and . . . ” and “ranging from . . . to . . . ” should be understood as meaning limits included, unless otherwise specified.

The invention is illustrated in more detail by the examples and figures presented below. Unless otherwise indicated, the amounts shown are expressed as percentages by weight.

### Example 1: Kit According to the Invention for Making Up the Eyelashes: Reducing Composition (A)₁ with 5% of L-Cysteine and Mascara (B)₁ of the Rocket Volum' Express® Type from Maybelline

Example 1 according to the invention of a kit for making up the eyelashes was carried out with the reducing composition (A)₁ with 5% of L-cysteine and the mascara (B)₁, which corresponds to a commercial mascara of the Rocket Volum' Express® range from Maybelline provided with its applicator.

TABLE 1

| | Reducing |
|---|---|
| Ingredients (INCI name) | composition (A)₁ |
| Aminomethylpropanol | 3.5 |
| L-Cysteine (Ajinomoto) | 5.0 |
| Hydroxyethylcellulose | 1.0 |
| (Cellulosize HEC QP ® from | |
| Amerchol) | |
| Water | q.s. for 100 |

### Preparation Protocol

### Protocol for the Preparation of the Composition (A)

The hydroxyethylcellulose is dispersed in water at 60° C. until a homogeneous solution is observed. The composition is subsequently cooled in order to subsequently add thereto the remainder of the ingredients with stirring.

The mascara (B)₁ of the Rocket Volum' Express® range from Maybelline has as complete list of the ingredients:
Aqua (Water), PEG-200 Glyceryl Stearate, Styrene/Acrylates/Ammonium Methacrylate Copolymer, Cera Alba (Beeswax), Paraffin, Copernicia Cerifera Cera (Carnauba Wax), Butylene Glycol, Stearic Acid, Cetyl Alcohol, Palmitic Acid, Ethylenediamine/Stearyl Dimer Dilinoleate Copolymer, VP/Eicosene Copolymer, Lithium Magnesium Sodium Silicate, Dimethicone, Aminomethyl Propanediol, Phenoxyethanol, Silica, Caprylyl Glycol, Sodium Dehydroacetate, Tocopheryl Acetate, Sodium Laureth Sulfate, Dimethiconol, Disodium EDTA, Polyurethane-35, Tetrasodium EDTA, +/− May Contain (Iron Oxides (CI 77491, CI 77492, CI 77499), CI 77007 (Ultramarines), CI 77891 (Titanium Dioxide), Mica, CI 75470 (Carmine), CI 77288 (Chromium Oxide Greens), CI 77289 (Chromium Hydroxide Green), CI 77742 (Manganese Violet), CI 77510 (Ferric Ferrocyanide)), F.I.L. D54745/1, G68392111.
   The mascara (B)₁ comprises:
an aqueous phase: Water;
a fatty phase with waxes: Cera Alba (Beeswax), Paraffin, Copernicia Cerifera Cera (Carnauba Wax), Cetyl Alcohol;
at least one anionic surfactant: Stearic Acid, Palmitic Acid associated with a cationic counterion: Aminomethyl Propanediol;
at least one film-forming polymer in the form of solid particles in suspension: Styrene/Acrylates/Ammonium Methacrylate Copolymer, Polyurethane-35.

### Example 2: Kit for Making Up the Eyelashes According to the Invention: Reducing Composition (A)₁ with 5% of L-Cysteine and Mascara (B)₂ of the Volume Effet Faux Cils® Type from Yves Saint Laurent

Example 2 according to the invention of a kit for making up the eyelashes was carried out with the composition (A)₁ as defined above with 5% of L-cysteine and the mascara (B)₂ below, which corresponds to a commercial product of the Volume Effet Faux Cils® range from Yves Saint Laurent provided with its applicator.

The mascara (B)₂ of the Volume Effet Faux Cils® range from Yves Saint Laurent has as complete list of the ingredients:
Water, Paraffin, Stearic Acid, Palmitic Acid, Copernicia Cerifera Cera/Carnauba Wax, C₁₀-30 Cholesterol/Lanosterol Esters, Cera Alba (Beeswax), Triethanolamine, Stearyl Stearate, Silica, Nylon-12, Aminomethyl Propanol, Phenoxyethanol, Hydroxyethylcellulose, Candelilla Cera (Candelilla Wax), Dimethicone, Myristic Acid, Tocopheryl Acetate, Panthenol, Methylparaben, VP/Eicosene Copolymer, Adipic Acid/Diethylene Glycol/Glycerin Crosspolymer, Sorbitan Stearate, Butylparaben, PEG-40 Stearate, BHT, Behenic Acid, Ethylparaben, Argania Spinosa Oil (Argania Spinosa Kernel Oil), Ricinus Communis (Castor) Seed Oil, Prunus Amygdalus Dulcis Oil (Sweet Almond Oil), Propylparaben, Carthamus Tinctorius Seed Oil (Safflower Seed Oil), Benzyl Alcohol, Fragrance.
   The mascara (B)₁ comprises:
an aqueous phase: Water;
a fatty phase with waxes: Paraffin, Copernicia Cerifera Cera/Carnauba Wax, Cera Alba (Beeswax), Candelilla Cera (Candelilla Wax);
at least one anionic surfactant: Stearic Acid, Palmitic Acid associated with a cationic counterion: Aminomethyl Propanediol, Triethanolamine.

### Example 3: Kit for Making Up the Eyelashes Outside the Invention: Reducing Composition (A)₁ with the Mascara (B)₃ of the Volume Effet Faux Cils Waterproof® Type from Yves Saint Laurent

Example 3 outside the invention of a kit for making up the eyelashes was carried out with the reducing composition (A)₁ with 5% of L-cysteine as defined above and the anhydrous mascara (B)₃, which corresponds to a commercial product of the Volume Effet Faux Cils Waterproof® range from Yves Saint Laurent provided with its applicator.

The anhydrous mascara (B)₃ of the Volume Effet Faux Cils Waterproof® range from Yves Saint Laurent has as complete list of the ingredients: Isododecane, Cera Alba/Beeswax, Copernicia Cerifera Cera/Carnauba Wax, Disteardimonium Hectorite, CI 77499/Iron Oxides, Aqua/Water, Allyl StearateNA Copolymer, Oryza Sativa Cera/Rice Bran Wax, Paraffin, Alcohol Denat, Polyvinyl Laurate, VP/Eicosene Copolymer, Propylene Carbonate, Talc, Synthetic Beeswax, Ethylenediamine/Stearyl Dimer Dilinoleate Copolymer, PEG-30 Glyceryl Stearate, Parfum/Fragrance, Tocopheryl Acetate, Candelilla Cera/Candelilla Wax, Panthenol, Argania Spinosa Kernel Oil, Ricinus Communis Seed Oil/Castor Seed Oil, Prunus Amygdalus Dulcis Oil/Sweet Almond Oil, Carthamus Tinctorius Seed Oil/Safflower Seed Oil, Benzyl Alcohol, Glycerin, Pentaerythrityl Tetra-di-t-butyl Hydroxyhydrocinnamate, BHT, Juglans Regia Leaf Extract/Walnut Leaf Extract.

### Example 4: Kit for Making Up the Eyelashes Outside the Invention: Composition (A)₂ without L-Cysteine and Mascara (B)₁ of the Rocket Volum' Express® Type from Maybelline

Example 4 outside the invention of a kit for making up the eyelashes was carried out with the composition (A)₂ without L-cysteine as defined below and the mascara (B)₁ as defined above provided with its applicator.

TABLE 2

| | Composition (A)₂ |
|---|---|
| Ingredients (INCI name) | without cysteine |
| Aminomethylpropanol | 3.5 |
| pH = 8.5 | |
| Hydroxyethylcellulose | 1.0 |
| (Cellosize HEC QP ® from | |
| Amerchol) | |
| Water | q.s. for 100 |

### Example 5: Kit for Making Up the Eyelashes Outside the Invention: Composition (A)₂ without L-Cysteine and Mascara (B)₂ of the Volume Effet Faux Cils® Type from Yves Saint Laurent

Example 5 outside the invention of a kit for making up the eyelashes was carried out with the composition (A)₂ without L-cysteine as defined above and the mascara (B)₂ as defined above provided with its applicator.

### Example 6: Kit for Making Up the Eyelashes Outside the Invention: Composition (A)₂ without L-Cysteine and Anhydrous Mascara (B)₃ of the Volume Effet Faux Cils Waterproof® Type from Yves Saint Laurent

Example 7 outside the invention of a kit for making up the eyelashes was carried out with the composition (A)₂ without L-cysteine as defined above and the anhydrous mascara (B)₃ as defined above provided with its applicator.

### Comparative Tests for Measuring the Curvature and the Durability

The six kits 1 to 6 for making up the eyelashes were compared, which comparison was carried out on a double test specimen of false eyelashes made of yak hairs “Yak Belly 2.7 g/20 cm Tips Not Cut Without String And Label®” from International Hair Importers & Products Inc.

The compositions (A)ᵢ and the compositions (B)ᵢ of each kit were successively applied to a double test specimen of false eyelashes.

Each composition (A)ᵢ was applied to the false eyelashes using a cotton swab by two series of 15 applications. The false eyelashes, thus treated, were left standing for 2 minutes and then the surplus of composition (A)ᵢ was wiped off with a paper towel.

The composition (B)₁ was applied to the false eyelashes by two series of 15 strokes using the applicator of the mascara of the Rocket Volum' Express® range from Maybelline.

The composition (B)₂ was applied to the false eyelashes by two series of 15 strokes using the applicator of the mascara of the Volume Effet Faux Cils® range from Yves Saint-Laurent.

For each kit 1 to 6, after a first making up with successively the application of the composition (A)ᵢ and of the composition (B)ᵢ, the made up test specimens were left for at least 6 hours and then the makeup was removed from the false eyelashes with a micellar makeup-removing solution from Garnier. The making up of the false eyelashes with the compositions (A)ᵢ and (B)ᵢ was then repeated successively twice, followed by a stage of makeup removal, over three consecutive days. Subsequently, for each makeup kit 1 to 6, the effect of curving of the eyelashes and the durability of the effect after a first makeup operation, then first makeup removal, then two successive further operations of making up and removing the makeup, were observed with the naked eye. The results obtained are shown in the following table 3.

TABLE 3

| | | Kit 3 | Kit 4 | Kit 5 | Kit 6 |
|---|---|---|---|---|---|
| Kit 1 | Kit 2 | (outside the | (outside the | (outside the | (outside the |
| (invention) | (invention) | invention) | invention) | invention) | invention) |
| (A)₁ | (A)₁ | (A)₁ | (A)₂ | (A)₂ | (A)₂ |
| 5% of | 5% of | 5% of | without | without | without |
| L-cysteine | L-cysteine | L-cysteine | cysteine | cysteine | cysteine |
| Mascara (B)₁ | Mascara (B)₂ | Anhydrous | Mascara (B)₁ | Mascara (B)₂ | Mascara (B)₃ |
| Rocket | Volume Effet | mascara (B)₃ | Rocket | Volume Effet | Volume Effet |
| Volum' | Faux Cils ® | Volume Effet | Volum' | Faux Cils ® | Faux Cils |
| Express ® | (Yves Saint | Faux Cils | Express ® | (Yves Saint | Waterproof ® |
| (Maybelline) | Laurent) | Waterproof ® | (Maybelline) | Laurent) | (Yves Saint |
| | | (Yves Saint | | | Laurent) |
| | | Laurent) | | | |
| Significant | Significant | No curving | No curving | No curving | No curving |
| curving of the | curving of the | persistent to | persistent to | persistent to | persistent to |
| eyelashes and | eyelashes and | makeup- | makeup- | makeup- | makeup- |
| persistent to | persistent to | removal | removal | removal | removal |
| makeup- | makeup- | operations | operations | operations | operations |
| removal | removal | | | | |
| operations | operations | | | | |

It has been found that the kit examples 1 and 2 according to the invention comprising an aqueous composition (A), having a pH>8.0 comprising at least 1% of L-cysteine (i.e. 5%) and a mascara (B), or (B)₂ comprising an aqueous phase, a fatty phase comprising at least one wax, resulted in a significant curving of the eyelashes which is durable and persistent to the 3 successive makeup-removal operations, unlike
the kit 3 outside the invention comprising an aqueous composition (A)₁ having a pH>8.0 comprising at least 1% of L-cysteine (i.e. 5%) and an anhydrous mascara (B)₃
the kits 4 to 6 outside the invention, in which the composition (A)₂ does not contain L-cysteine.

### Examples 7 and 8 (Outside the Invention): Mascaras with 0.5% Cysteine or without Cysteine

TABLE 4

| | | | | Example 8 |
|---|---|---|---|---|
| | | | Example 7 | (Outside the |
| | | Commercial | (Outside the | invention, |
| Ingredients | Supplier | name | invention) | comparative) |
| Di sodium EDTA | BASF | EDETA BD | 0.2 | 0.2 |
| Iron oxides | Sun | SunPURO BLACK | 7.14 | 7.14 |
| | | IRON OXIDE C33- | | |
| | | 7001 | | |
| Sodium dehydroacetate | Shanghai | Sodium | 0.3 | 0.3 |
| | Xinwang | dehydroacetate | | |
| | Polymer | | | |
| | Material | | | |
| Phenoxyethanol | SEPPIC | Sepicide LD | 0.84 | 0.84 |
| Carnauba wax | Strahl & Pitsch | Carnauba Wax #1 | 3.5 | 3.5 |
| (Copernicia Cerifera Wax) | | Flakes N.F. SP 63 | | |
| Beeswax (Beeswax) | Koster Keunen | White Beeswax | 4.4 | 4.4 |
| | | (GR B 889) | | |
| Cetyl alcohol | BASF | Lanette 16 | 2 | 2 |
| Paraffin | Baerlocher | Affine 56-58 | 11.12 | 11.12 |
| | | Pastilles | | |
| Hydrogenated vegetable oil | Desert Whale | Jojoba Wax Flakes | 0.25 | 0.25 |
| Hydrogenated palm oil | SIO (ADM) | GV 60 | 0.25 | 0.25 |
| Hydroxyethylcellulose | Amerchol (Dow | Cellosize QP 4400 | 0.75 | 0.75 |
| | Chemical) | H | | |
| Acacia gum | Nexira | Spraygum SC10 | 0.63 | 0.63 |
| (Acacia Senegal Gum) | | | | |
| Ethylene/acrylic acid copolymer | Honeywell | Asensa SC 401 | 2.78 | 2.78 |
| Acrylates copolymer as 50% | Daito Kasei | Daitosol 5000 AD | 10 | 10 |
| aqueous dispersion | Kogyo | | | |
| Ethylenediamine/stearyl | Croda | Oleocraft LP-10- | 0.5 | 0.5 |
| dimer dilinoleate copolymer | | PA-(MV) | | |
| Water | | | q.s. for 100 | q.s. for 100 |
| Caprylyl glycol | Dr Straetmans | Dermosoft Octiol | 0.3 | 0.3 |
| Steareth-2 | Evonik | Tego Alkanol S2 | 2.1 | 2.1 |
| | Goldschmidt | | | |
| Potassium cetyl phosphate | DSM | Amphisol K | 5 | 5 |
| | Nutritional | (0452130) | | |
| | Products | | | |
| Cysteine | Ajinomoto | L-Cysteine | 0 | 0.5 |

The two formulas are packaged in the Hypnose® pack from Lancôme®. The products were applied to test specimens of false eyelashes made of yak hairs. These mascaras are applied to the test specimens of false eyelashes by depositing two times 15 strokes of brushes. The mascara is left on the test specimens for 6 hours and then makeup is removed from the test specimens with a makeup-remover of micellar water type from Garnier. The curvature of the test specimens of false eyelashes is subsequently evaluated. It is found that there is no difference between the compositions of examples 7 and 8, which give no curving of the eyelashes. There is thus no effect observed for 0.5% cysteine in a mascara composition on the modification of the curvature of a keratin fiber.

1: A kit for making up keratin fibers, comprising:
a) a first composition (A) comprising at least 40.0% by weight of water, with respect to the total weight of the composition, and at least 1.0% by weight, with respect to the total weight of the composition, of cysteine and/or one of its salts, said composition having a pH of greater than 8.0, and
b) a second composition (B) comprising:
i) an aqueous phase,
ii) a fatty phase comprising at least one wax,
iii) at least one anionic surfactant comprising at least one cationic counterion; and
iv) optionally at least one film-forming polymer in the form of solid particles in suspension in the aqueous phase of the composition (B).

2: The kit as claimed in claim 1, where the cysteine is L-cysteine or one of its salts.

3: The kit as claimed in claim 1, where the cysteine and/or one of its salts is present in the composition (A) at a concentration ranging from 1.5% to 8% by weight, with respect to the total weight of the composition (A).

4: The kit as claimed in claim 1, where the pH of the composition (A) is from 8.5 to 10.0.

5: The kit as claimed in claim 1, where the amount of water in the composition (A) varies is from 50% to 95% by weight with respect to the total weight of the composition (A).

6: The kit as claimed in claim 1, where the composition (A) comprises at least one hydrophilic gelling agent.

7: The kit as claimed in claim 6, where the hydrophilic gelling agent(s) are present in the composition (A) in a total content ranging from 0.1% to 10% by weight, with respect to the total weight of said composition (A).

8: The kit as claimed in claim 1, where the composition (A) comprises one or more nonvolatile oils in a total concentration of less than 5% by weight, with respect to the total weight of the composition (A).

9: The kit as claimed in claim 1, where the composition (B) comprises water in a concentration ranging from 30% to 70% by weight with respect to the total weight of the composition (B).

10: The kit as claimed in claim 1, where the fatty phase of the composition (B) is at a content of between 5% and 45% by weight with respect to the total weight of the composition (B).

11: The kit as claimed in claim 1, where the wax(es) of the composition (B) are present in a content of at least 5% by weight with respect to the total weight of composition (B).

12: The kit as claimed in claim 1, where the anionic surfactant(s) are present in the composition (B) at a total content of greater than or equal to 2% by weight with respect to the total weight of the composition (B).

13: The kit as claimed in claim 1, where, in the composition (B), the anionic surfactant(s) are chosen from:
i. alkyl phosphates;
ii. alkyl sulfates;
iii. alkylsulfonates, alkylamidesulfonates, alkylarylsulfonates, α-olefinsulfonates, and paraffinsulfonates;
iv. alkyl sulfosuccinates, alkyl ether sulfosuccinates, alkylamide sulfosuccinates; and alkyl sulfosuccinamates;
v. alkyl sulfoacetates;
vi. acyl sarcosinates, acyl glutamates, acyl isethionates, N-acyl taurates, and acyl lactylates;
vii. alkylpolyglycoside carboxylic esters, alkylpolyglycoside sulfosuccinates, and alkylpolyglycoside sulfosuccinamates;
viii. fatty acids;
ix. alkyl-D-galactosiduronic acids;
x. polyoxyalkylenated (C6-C24)alkyl ether carboxylic acids, polyoxyalkylenated (C6-C24)alkylaryl ether carboxylic acids, and polyoxyalkylenated (C6-C24)alkylamido ether carboxylic acids; and
xi. their mixtures.

14: The kit as claimed in claim 1, where, in the composition (B), the cationic counterion(s) are chosen from a cation of inorganic origin.

15: The kit as claimed in claim 1, where, in the composition (B), the total content of cationic counterion(s) is greater than or equal to 0.01% by weight, with respect to the total weight of the composition (B).

16: The kit as claimed in claim 1, where, in the composition (B), the anionic surfactant is chosen from:
C₁₂-C₂₂, fatty acids;
alkyl phosphates; and
their mixtures.

17: The kit as claimed in claim 1, where the composition (B) comprises at least one film-forming polymer in the form of solid particles in suspension in the aqueous phase of the composition.

18: The kit as claimed in claim 1, where, in the composition (B), the film-forming polymer(s) are present in a total solids content ranging from 0.5% to 10% by weight with respect to the total weight of the composition (B).

19: The kit as claimed in claim 1, where the composition (B) comprises an aqueous dispersion of solid particles of film-forming polymer of styrene, of ammonium methacrylate and of a monomer chosen from acrylic acid, methacrylic acid or one of their simple esters.

20: The kit as claimed in claim 1, where the composition (B) comprises an aqueous dispersion of solid particles of film-forming polymer of Styrene/Acrylates/Ammonium Methacrylate Copolymer.

21: The kit as claimed in claim 1, where the composition (B) comprises at least one colorant.

22: The kit as claimed in claim 1, where the composition (B) is in the form of a mascara.

23: A cosmetic method for making up keratin fibers the eyelashes, comprising
at least one first stage of application of a first composition (A) as defined in any one of the preceding claims to said keratin fibers, and successively
at least one second stage of application of at least one second composition (B) as defined in any one of the preceding claims to said fibers treated by the composition (A).

24: The method as claimed in claim 23, in which, after application to the keratin fibers of the composition (A), there is a pause for a period of time of from 1 to 20 minutes before applying the composition (B).

25: The method as claimed in claim 23 or 24, in which the successive stages of application of the composition (A) and (B) are repeated on said fibers more than once.
